(19) United States
(12) Patent Application Publication
(10) Pub. No.: US 2009/0143781 A1
(43) Pub. Date: Jun. 4, 2009

(54) METHODS AND DEVICES FOR TREATMENT OF BONE FRACTURES

(76) Inventor: Hans A. Mische, St. Cloud, MN (US)

Correspondence Address: Hans A. Mische, 32 Highbanks Place, St. Cloud, MN, 56301, US

(21) Appl. No.: 12/258,309

(22) Filed: Oct. 24, 2008

Related U.S. Application Data
(63) Continuation-in-part of application No. 09/733,775, filed on Dec. 8, 2000.
(60) Provisional application No. 60/982,931, filed on Oct. 26, 2007.
(60) Provisional application No. 60/169,778, filed on Dec. 9, 1999.
(60) Provisional application No. 60/181,651, filed on Feb. 10, 2000.
(60) Provisional application No. 60/191,664, filed on Mar. 23, 2000.

(51) Int. Cl. A61B 17/58

(52) U.S. Cl. 606/63

(57) ABSTRACT

A method and devices for facilitating fixating and joining of bone fractures utilizing expandable devices that are positioned within the bone and across the fracture site. The stress from the expanded devices may enhance and expedite bone healing.

### CROSS-REFERENCE TO RELATED APPLICATIONS

This application claims the benefit of priority to U.S. Provisional No. 60/982,931, filed on Oct. 26, 2007, which is incorporated by reference in its entirety herein.

This application claims the benefit as a continuation-in-part of priority from U.S. application Ser. No. 09/733,775, filed Dec. 8, 2000, which claims priority to U.S. Provisional Nos. 60/169,778, filed Dec. 9, 1999, 60/181,651, filed Feb. 10, 2000, and 60/191,664, filed Mar. 23, 2000, all of which are incorporated by reference in their entireties herein.

### BACKGROUND OF THE INVENTION

1. Field of the Invention

Embodiments of the present invention relate generally to the treatment of bone fractures utilizing expandable fracture fixating devices within the medullary cavity of bones, and equipment and methods specially designed for implanting these devices. Novel methods and devices for the treatment of bone fractures are disclosed.

2. Description of the Related Art

The current methods of treating bone fractures range from simple setting of the bone and constraining motion via a cast or wrap to using pins, screws, rods and cement to fixate fracture site. With the use of casts, the bone is not stabilized and misalignment may occur after placing the cast. This may require the cast to be removed and the bone reset. This is a very uncomfortable and painful procedure for the victim or patient and can ultimately result in permanent misalignment of the healed bone. The treatment modalities requiring a surgical procedure are painfull and are associated with a high rate of complications. Post-procedural infections are one of the major complications associated with these surgical procedures. Many of these infections result in necrosis of bone and tissue and require additional surgical interventions and therapy. The invention discussed here provides for a unique and novel means of treating a variety of bone fractures with minimally invasive techniques and low complication rates.

### SUMMARY OF THE INVENTION

In contrast to the prior art, embodiments of the present invention propose treatment of bone fractures using minimally invasive techniques, methods, equipment and devices to position and deliver an expandable fracture fixating device into the medullary cavity (marrow conduit). The device is preferably an expandable structure that “bridges” the fracture site and fixates the site upon expansion. In addition to fixation, the device also joins the fractured bone such as in the case of a compound fracture. Referring to the device as a bridge, the BRIDGE is substantially hollow and has low surface area and mass, the majority of bone marrow volume can be preserved. The ability to preserve a large quantity of the bone marrow cavity is beneficial for healing, bone health and maintaining the body's natural ability to generate red blood cells. In addition, the stress applied to the bone by the expanded or expanding “BRIDGE” facilitates rapid bone growth and strength. The operable level of stress applied to the bone will vary from low levels to high levels dependent on the type, size and location of bone to be treated. It is also envisioned that the BRIDGE can be used to expand and support bones that are crushed or compressed. The BRIDGE can be delivered by a variety of expansion devices, can be self expanding to due to inherent spring forces within the BRIDGE structure, or can be expansively actuated utilizing elements and mechanisms within the BRIDGE structure. These various devices and alternative embodiments will be detailed further.

Although standard medical equipment may be used to facilitate the procedure, it may be necessary to design unique, specialized tools in order for this invention to be properly utilized. These devices may include tissue separators, retractors, drills, introducers, coring tools, and others.

The invention is disclosed in the context of treating bone fractures but other organs and anatomical tissues are contemplated as well. For example, the invention may be used to treat spinal stenosis, individual vertebrae, and support or fixate segments of the spinal column. Likewise, a broken nose, sinus cavity or collapsed lung can be supported using this invention. Pelvic fractures in females could also benefit from placing this device within the vaginal cavity in order to support and fixate the pelvis or pubic bone.

### BRIEF DESCRIPTION OF THE DRAWINGS

Throughout the several views of the drawings several illustrative embodiments of the invention are disclosed. It should be understood that various modifications of the embodiments might be made without departing from the scope of the invention. Throughout the views identical reference numerals depict equivalent structure wherein:

FIG. 1 is a diagram showing the advancement and deployment of the BRIDGE utilizing a catheter with an expandable element according to one embodiment of the present invention.

FIG. 2 is a diagram showing the advancement and deployment of a self-expanding BRIDGE according to one embodiment of the present invention.

FIG. 3 is a diagram showing the advancement and deployment of the BRIDGE, utilizing a catheter with an expandable element, within a compressed bone segment according to one embodiment of the present invention.

FIG. 4 shows a variety of acceptable BRIDGE structures and designs according to various embodiments of the present invention.

FIGS. 5A & B depict a bridge that can be expanded or contracted by relative movement of the ends of the structure FIG. 6 shows a bridge that can be expanded or contracted by relative movement of the ends of the structure according to one embodiment of the present invention.

FIG. 7 shows a bridge that can be expanded or contracted by relative movement of the ends of the structure according to one embodiment of the present invention.

FIGS. 8A & 8B show the placement of a coil BRIDGE according to one embodiment of the present invention.

FIGS. 9A & 9B show the placement of a braided BRIDGE according to one embodiment of the present invention.

FIG. 10 shows the screws or nails used in conjunction with an implanted BRIDGE according to one embodiment of the present invention.

FIG. 11 shows a BRIDGE used in conjunction with external supporting elements according to one embodiment of the present invention.

FIGS. 12A & 12B shows an implanted BRIDGE connected to an electrical generator according to one embodiment of the present invention.

FIG. 13 shows an expansion device using a rubber grommet according to one embodiment of the present invention.

FIG. 14A is a schematic front axial view of a distal anchor of a bone bridge according to one embodiment of the present invention.

FIG. 14B is a schematic front axial view of the distal anchor of a bone bridge in a deployed configuration according to the embodiment of FIG. 14A.

FIG. 15A is a schematic front axial view of a proximal anchor of a bone bridge according to one embodiment of the present invention.

FIG. 15B is a schematic front axial view of the proximal anchor of a bone bridge in a deployed configuration according to the embodiment of FIG. 14A.

FIG. 16 is a schematic side view of a bone bridge comprising a shaft, one or more anchors, and a retractable sheath according to one embodiment of the present invention.

FIG. 17 is a schematic side view of the bone bridge of FIG. 16 without the retractable shaft.

FIG. 18 is a schematic side view of a bone bridge comprising a generator according to one embodiment of the present invention.

FIG. 19A-C is a schematic side view of a bone bridge with two or more expandable features according to one embodiment of the present invention.

While the invention will be described in connection with the preferred embodiment, it will be understood that it is not intended to limit the invention to that embodiment.

### DETAILED DESCRIPTION OF THE PREFERRED EMBODIMENT

Throughout the description the term BRIDGE refers to an expandable device that is used to fixate or repair bone fractures. The device may be made of metals such as stainless steel, tantalum, titanium, Nitinol or Elgiloy and it may form an electrode for electrical stimulation. One or more electrodes may be associated with it. The BRIDGE may incorporate fiber optics for imaging, sensing, or the transmission of energy to heat, ablate, illuminate, or as one skilled in the art would understand, cure therapeutic materials such as polymers and adhesive. The device may also be made from a plastic or other non-metallic material. The BRIDGE may also incorporate a covering of polymer or other materials. The BRIDGE may also be a composition of different materials. The BRIDGE may be smooth or have cutting or abrasive surfaces. The BRIDGE can be self-expanding or use a device such as a balloon catheter to mechanically expand or further expand it. In addition, other means of expanding the BRIDGE may be utilized such as any mechanical means of expansion, or thermal, vibrational, electrical, hydraulic, pneumatic actuation. Mechanical means might employ a system consisting of a rubber grommet that expands when it is compressed axially. Another mechanical means of expansion may use a tubular array of elements such as splines, wires or braided wire that expand radially outward when compressed at each end. Another mechanical means could employ wedges in a tubular or cylindrical type of array that collectively force the BRIDGE to expand when they are moved relative to each other. The BRIDGE delivery system may also employ fiber optic technology in order to endoscopically diagnose, control placement and review procedural outcome. In addition, in one embodiment the fiber optics of the delivery system could also be used to deliver light energy such as UV and IR, to cure therapeutic materials such as polymers and adhesive. Likewise, a number of other technologies such as pressure monitoring, stress monitoring, volume monitoring, etc. can be employed to benefit the outcome of the procedure.

The BRIDGE may be implanted for chronic use or for acute use. In acute use, the BRIDGE is used for temporary stabilization and fixation of bone fractures. After a period of time, the BRIDGE is withdrawn.

Biodegradable materials that degrade or dissolve over time may be used to form the BRIDGE. Various coatings may be applied to the BRIDGE including, but not limited to, thrombo-resistant materials, electrically conductive, non-conductive, thermo-luminescent, heparin, radioactive, or biocompatible coatings. Materials such as calcium, minerals, or irritants can be applied to the BRIDGE in order to expedite bone growth. Drugs, chemicals, and biologics such as morphine, dopamine, aspirin, genetic materials, antibiotics and growth factors can be applied to the BRIDGE in order to facilitate treatment.

Other types of additives can be applied as required for specific treatments.

Electrically conductive BRIDGEs with electrode elements may be used with companion pulse generators to deliver stimulation energy to the bone to expedite bone growth. This electrical therapy may be used alone or in combination with other therapies to treat the affected site. Electrical therapies may be supplied from implantable devices or they may be coupled directly to external generators. Coupling between the BRIDGE and external generators can be achieved using technologies such as inductive or microwave coupling as examples. The BRIDGE may also be designed of geometries or materials that absorb radioactive energies for the treatment of bone cancer, as an example.

In the preferred embodiment, access is gained to a location on the bone that the device will pass through. A surgical incision is made through tissue to expose the entry site at the bone. The size and scope of the incision is dependent on the need for each case, Preferably, a small hole is drilled through the bone into the medullary cavity (marrow conduit). Larger holes or removal of a portion of the bone may be required dependent on the need for each case.

In the example of a fractured femur, an access location might be the either the greater trochanter or the patellar surface. In the case of a fractured humerus, the access might be made at the greater tubercle or the capitulum

The device, on its delivery system, is then passed through the marrow cavity and positioned across the fracture.

When the right position is attained (potentially guided by CAT scan, MRI, x-ray, or fluoroscopic imaging), the fracture can be manipulated to an optimum configuration if needed, and the device is expanded or released for expansion. The delivery system is then removed after expansion.

If necessary, the access hole in the bone can be plugged with retained bone chips from the drilling procedure, fibrin or other acceptable materials.

Any surgical incision is closed with standard techniques.

It may be necessary to remove some bone marrow to facilitate placement of the BRIDGE. After placement of the BRIDGE, the marrow can be reinserted into the bone and within the BRIDGE. Another alternative treatment may be to replace the marrow with a polymeric substance that hardens after placement within the bridge and bone portions. This would enhance the immediate fixation strength. The polymeric substance can be biodegradable or otherwise metabolized by the body. In addition, the polymeric substance may incorporate drugs, antibiotics other clinically relevant substances and materials. The polymeric substance can also form a foam or cellular structure to allow for marrow formation. In various embodiments the polymeric substance can be made of a composition that hardens or polymerizes with the application of ultraviolet light, heat, radiation, electrical energy, or moisture as examples.

Other embodiments of the BRIDGE invention can include the use of external screws that join the BRIDGE through the bone. This provides and extra measure of securement and strength.

FIG. 1A is a diagram showing the BRIDGE 10, which is mounted to a balloon catheter delivery device 11 within a segment of fractured bone 12. The entire system is advanced through an opening 13 in the bone 12. The BRIDGE 10 is positioned to span the fracture. At this point, the balloon is inflated causing the BRIDGE to expand against the inside of the bone. The balloon may be inflated via a syringe or pump 14 and a pressure gauge 15. The balloon may have a pre-determined minimum or maximum diameter. In addition, the balloon can have a complex shape to provide proper placement and conformance of the device based on anatomical requirements and location. One or more inflations may be used to insure proper positioning and results. FIG. 1B shows the expanded BRIDGE 10 spanning the fracture and connecting the bone segments. The delivery device 11 is being withdrawn. If required, the balloon may be reinserted and reinflated for additional BRIDGE manipulation.

FIG. 2A is a diagram showing a self-expanding BRIDGE 20, which is compressed and inserted within a catheter delivery device 21, within a segment of fractured bone 22. The entire system is advanced through an opening 23 in the bone 22. The BRIDGE 20 is positioned to span the fracture.

At this point, the BRIDGE 20 is released from the catheter and self-expands against the inside of the bone. The release mechanism can be simply pushing the BRIDGE out of a catheter lumen or retracting a retaining sleeve. The BRIDGE self-expands due to the spring forces inherent in its materials and design. Likewise, the BRIDGE can be made of a shape-memory material such as Nitinol so that when subjected to body temperature the structure expands. With shape memory materials, the shape of the expanded device can be predetermined. Additionally, the device can be retrieved, repositioned, or removed by using temperature-based shape-memory characteristics.

FIG. 2B shows the expanded BRIDGE 20 spanning the fracture and connecting the bone segments. The delivery catheter 21 is being withdrawn.

In the self-expanding case, the tubular mesh has a predetermined maximum expandable diameter.

FIG. 3A shows a BRIDGE 30 on a balloon catheter 31 being advanced into a crushed area of a bone.

FIG. 3B shows the BRIDGE 30 expanded within the crush zone causing the crushed bone to resume its original diameter. The same results can be attained using any of the aforementioned BRIDGE designs, such as self-expanding or manually expanded, and placement methods. In the case of self-expanding designs, further expansion of the BRIDGE can be performed using a balloon catheter or another type of expansion device such as those mentioned within this invention or can use solid dilator rods.

FIG. 4 shows a variety of possible BRIDGE shapes and geometries. A tubular mesh 42, a multi-element spline 44, a coil 46, slotted tube 48, and a clam-shell or sleeve 49. In the case of slotted tube, other geometric configurations of the slots (i.e.; hexagonal, sinusoidal, circular, meandering, spiraling, and multigeometric patterns) may be utilized alone or in conjunction with a combination thereof Likewise, variations in the geometry of any of the BRIDGEs may be altered to achieve desired performance criteria such as radial strength, longitudinal flexibility or stiffness, expansion ease, profile, surface area, mass and volume, and material selection. The elements of the BRIDGE may be porous, have through holes, or have a covering. In addition, the surface of the bridge may be textured, rough, and sharp or have cleats or small pins integrated or attached. Each of the various shapes and geometries may find its own specialized use in the treatment of specific type of bone fractures.

FIG. 5 shows two states of a manually expandable BRIDGE device 51. The device consists of a coaxial shaft 52 and tube 53 arrangement. Attached to the distal end of the shaft 52 and the tube 53 is a braided mesh tube BRIDGE 51. When the shaft 52 and tube 53 are moved opposite of the other by manipulating the proximal ends, the BRIDGE 51 expands 54 or contracts 55. In this case, the BRIDGE 51 can be made of any structure that expands and contracts such as a coil, splined-elements, etc. The various methods of expanding and contracting these structures are, but not limited to, push-pull, rotation, and balloon manipulation. In this type of device, direct connection to either an electrical generator, laser, or monitoring system can be made. In addition, it be envisioned that a device of similar nature be connected to a mechanical energy source, such as rotational or vibrational sources.

FIG. 6 shows a manually expanded BRIDGE 60 with an internal rod 61 and compression nut mechanism 62. One end of the BRIDGE is fixed to one end of the rod 63, while the other end 64 is allowed to move relative to the rod. As the compression nut is tightened, it forces the end 64 of the BRIDGE to move, thus compressing the BRIDGE and forcing it to expand. Using a customized tool, the compression nut is tightened and the BRIDGE expanded until the desired affect is achieved. The nut can have a locking mechanism, such as a lock washer or other means, to maintain position. Alternatively, the nut and rod components can be exchanged for a bolt and nut or a bolt and internally threaded tubular rod. In any event, the expansion is caused by the relative movement of a a screw threaded mechanism.

FIG. 7 shows another manually expanded BRIDGE 70 with an internal rod 71 and compression element 72. One end of the BRIDGE is fixed to one end of the rod 73, while the other end 74 is allowed to move relative to the rod. As the compression element is pushed forward, it forces the end 74 of the BRIDGE to move, thus compressing the BRIDGE and forcing it to expand. The compression element is advanced and the BRIDGE expanded until the desired affect is achieved. The element can maintain its position utilizing mechanical friction or a detent mechanism. Other means of maintaining position are possible. The internal rod of the manually expanded BRIDGEs may be flexile or rigid. The expanding elements of the manually expanded BRIDGEs may utilize geometries such as those discussed in FIG. 4

FIGS. 8A & 8B show the use of a coil BRIDGE. The coil BRIDGE 81 is advanced to the fracture in a stretched state with a diameter less than its natural, unstretched diameter. When it is released from the delivery device 82, the coil BRIDGE expands to a state of greater diameter. As it expands to a greater diameter 83 it naturally shortens in length. This simultaneously draws the fracture together and fixates the fracture.

FIGS. 9A & 9B show the use of a braid BRIDGE. The braid BRIDGE 91 is advanced to the fracture in a stretched state with a diameter less than its natural, unstretched diameter. When it is released from the delivery device 92, the braid BRIDGE 93 expands to a state of greater diameter. As it expands to a greater diameter it naturally shortens in length. This simultaneously draws the fracture together and fixates the fracture. The devices in FIGS. 8 and FIG. 9 can utilize other geometries that function similarly with similar results. In addition, shape memory materials that exhibit similar change of length and diameter may be used in the construction of devices in FIG. 8 and FIG. 9.

FIG. 10 shows the BRIDGE 100 invention including the use of external screws 101 that join the BRIDGE through the bone. This provides an extra measure of securement and strength.

FIG. 11 shows external plates 10 incorporated with this combination of external screws 111 and BRIDGE 112. There maybe fractures that require the additional stabilization that this combination provides.

FIG. 12A shows an implanted bridge 120 connected to an electrical generator 121 in order to expedite bone growth. The external screws in FIG. 10 can serve the dual purpose of adding securement and acting as electrodes 122.

FIG. 12B shows a device 123 similar to that in FIG. 5 that is connected to an electrical generator 124. In this scenario, the BRIDGE can be used is in a temporary or permanent fashion. It may be desirable to remove the BRIDGE after the bone has healed.

FIG. 13 shows an expansion device 130 that uses a rubber sleeve or grommet 131 that when compressed axially 132, expands radially 133.

In some embodiments, discrete electrodes can be positioned on the bone bridge to facilitate electrical stimulation and resultant expedited bone growth and healing of fracture site. The electrodes can be placed at each end of the bridge or at various locations along the length and circumference. Electrodes may be spiraling, straight, circular or any other geometry along the length of the bridge. Electrodes may be monopolar, bipolar or multipolar designs.

In some embodiments, vibrational energy applied to the bone bridge, as disclosed in the applications incorporated by reference above and describe herein, can facilitate improved distribution of bone cements and adhesive. Vibrational energy can reduce the amount of air bubbles within the cements and adhesives to improve structural strength. In addition, the vibrational energy may drive or embedded the cements and adhesives into the pores, crevices, and fractures on or near the intramedullary surfaces. Likewise, the vibrational energy can help distribute the cements and adhesive amongst the bone bridge-intramedullary matrix in order to optimize strength and therapeutic parameters. Also, the delivery of drugs, stem cells, and other therapeutic substances can be performed in the similar manner.

In some embodiments, a bone bridge includes two structures, independently or cooperatively operated, that assist in the apposition of the fracture components of the bone. As illustrated in FIGS. 16 & 17, the bone bridge 160 comprises at least two separable structures that are positioned coaxially to each other. The structures, when manipulated, can move longitudinally respectively and independently of each other. Each structure has one or more element(s) that expands or projects radially when activated in various fashions. The expanding elements, when activated, grab, lock, embed, or attach the intramedullary surface. Upon the relative movement of the two structures, and as needed, the fracture site can be drawn closer together or farther apart in order to create the optimum apposition or contact of the fracture site. After this step, the two structures can be joined or fixated together in order to prevent movement. This can be done at the proximal ends via locking or attachment mechanisms that include, but not limited too, a lock nut/thread shaft arrangement, cotter pins, clasps, detents, compression fittings, etc.

In one embodiment, illustrated at FIGS. 16 and 17, a bone bridge 160 comprises a distal anchor shaft 161, a proximal anchor shaft 165 and a retractable sheath 169. The distal anchor shaft 161 is connected to a distal anchor 162 at a distal anchor interface 164. Distal anchor 162 is connected to one or more distal anchor elements 163, which expand outwardly radially when deployed, as illustrated in FIGS. 14A and 14B. As illustrated, four anchor elements 163 are attached, but one, two, three, four or more anchor elements can be used. The proximal anchor shaft 165 is connected to one or more proximal anchor elements 166, which expand outwardly radially when deployed, as illustrated in FIGS. 15A and 15B. As illustrated, four anchor elements 166 are attached, but one, two, three, four or more anchor elements can be used. In one embodiment, the proximal anchor shaft 165 is tubular. When the retractable sheath 169 is retracted, as illustrated in FIG. 17, the distal anchor 162 deploys and grabs the intramedullary surfaces. With continued retraction, the proximal anchor 166 deploys and grabs the intramedullary surface. With relative movement of the distal anchor shaft 161 and the proximal anchor shaft 165, as illustrated with arrows 170 and 172, apposition of the bone fracture can be controlled. Shafts can b e locked together to ensure permanent or temporary positioning. In one embodiment, the distal anchor shaft 161 and the proximal anchor shaft 165 can move with internal and external screw threads on each shaft. In one embodiment the device can also be used to position, move and/or fixate one or more vertebrae within the spinal column.

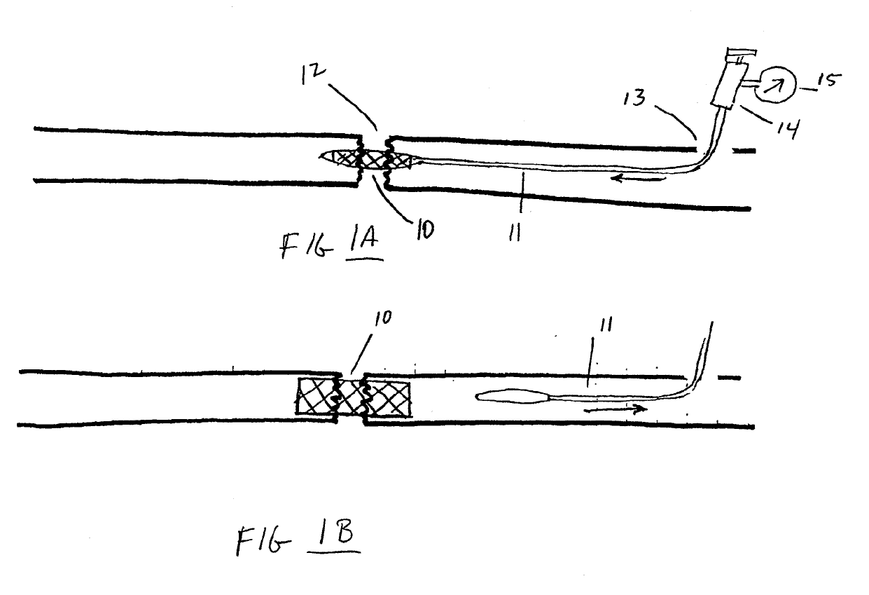

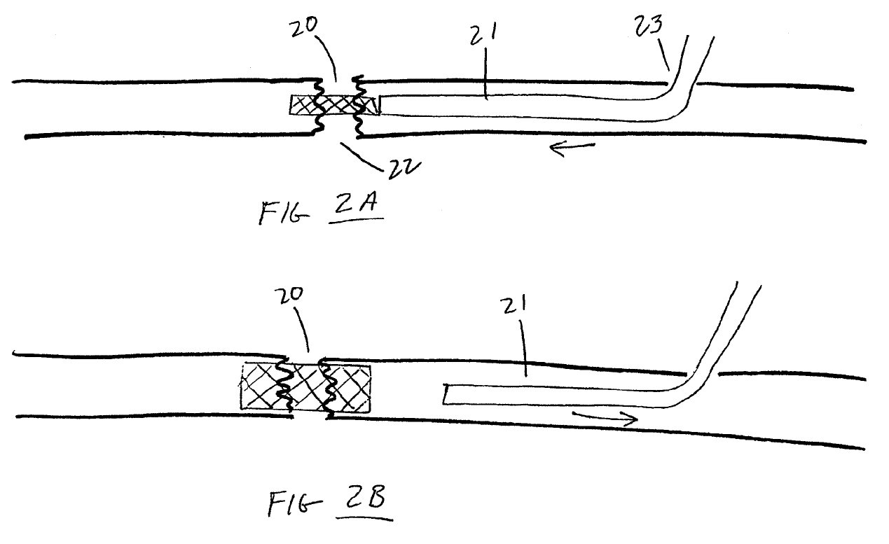

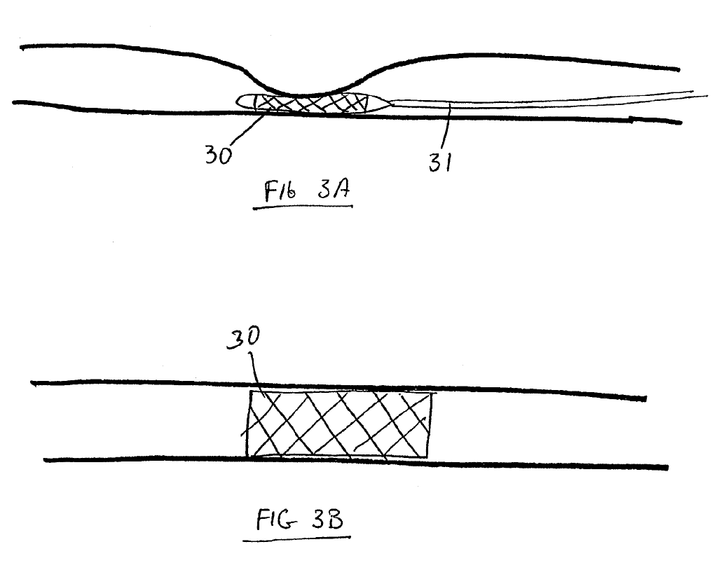

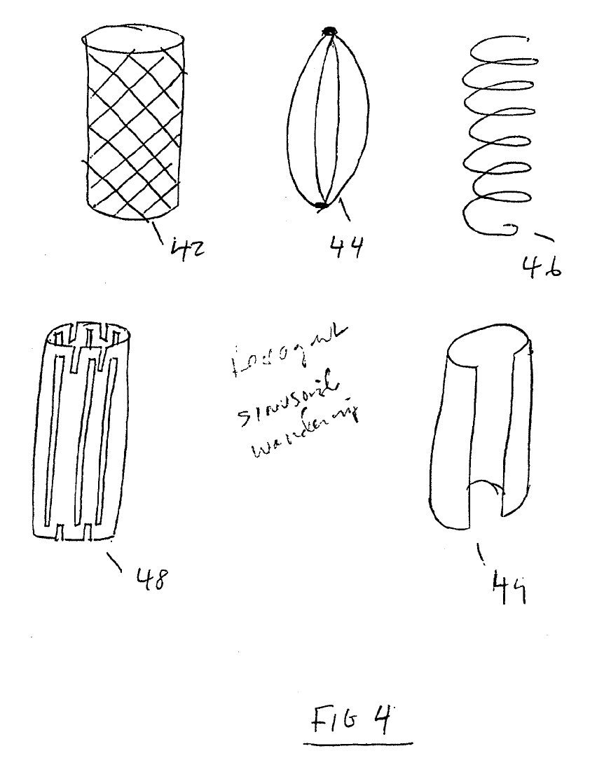

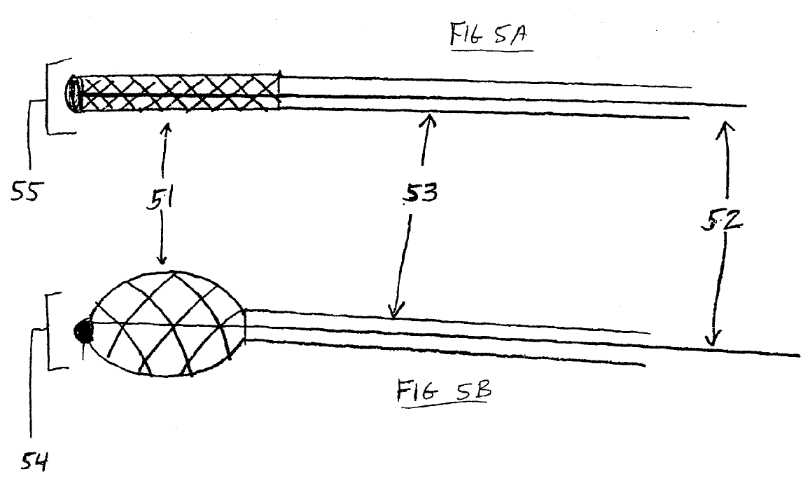

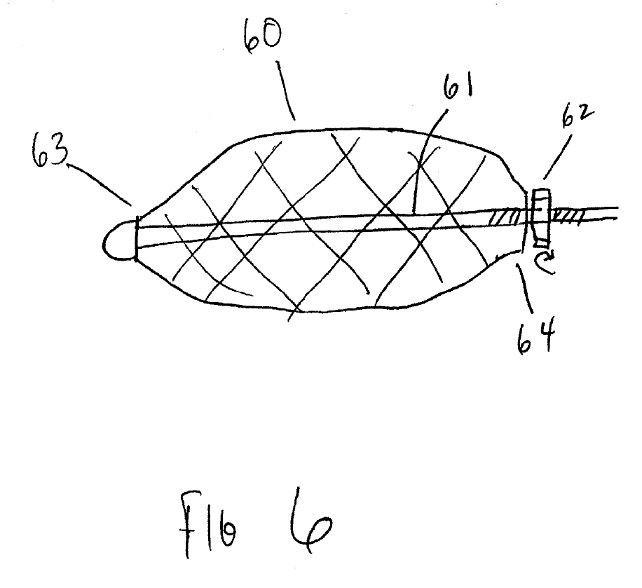

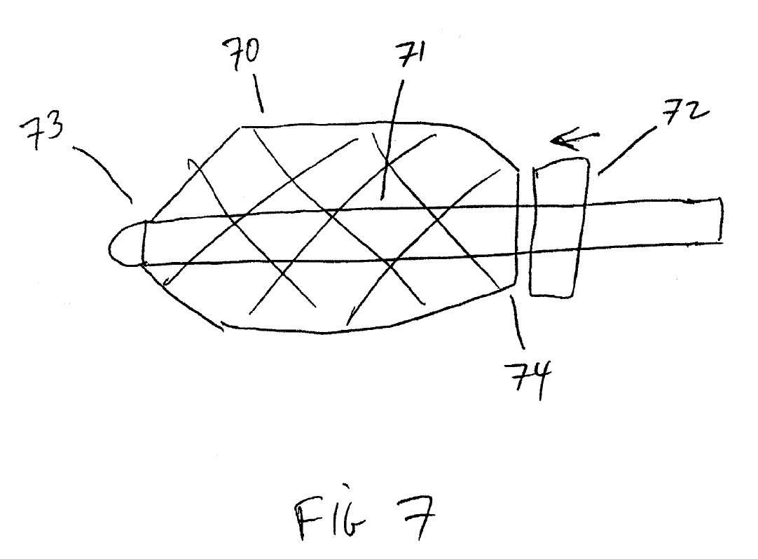

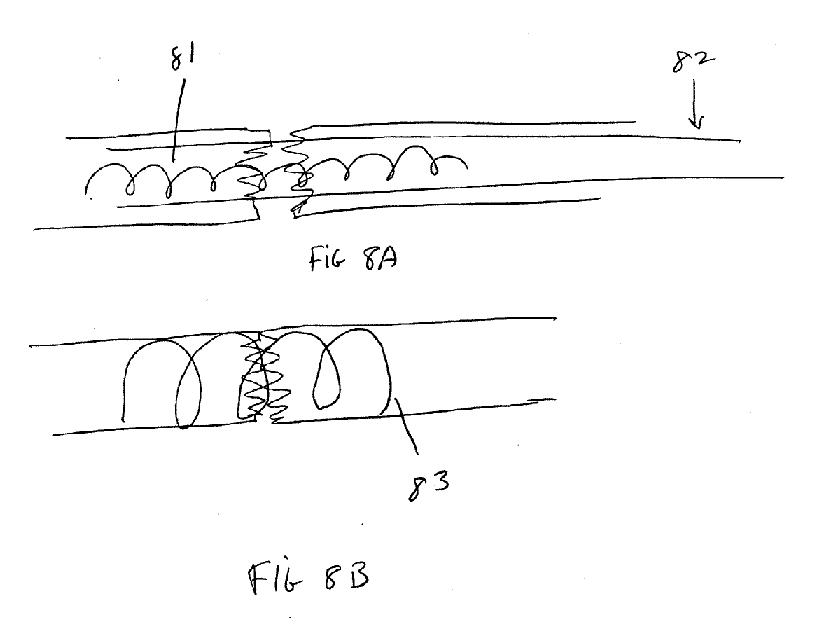

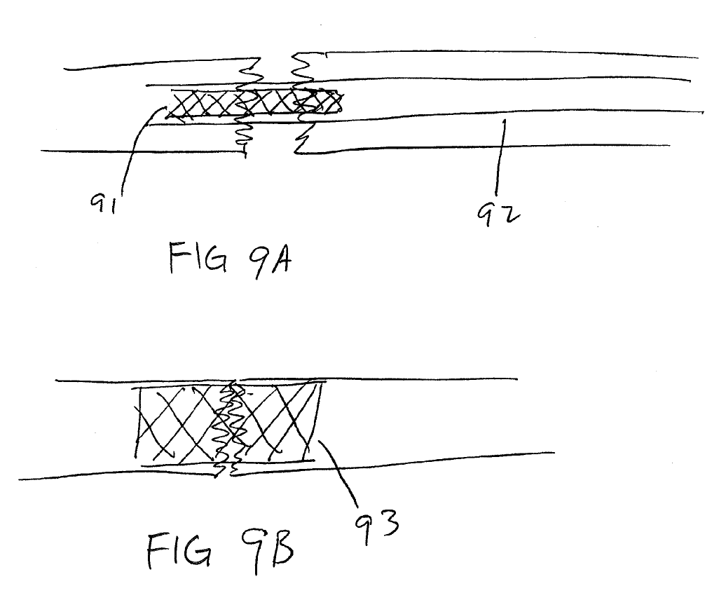

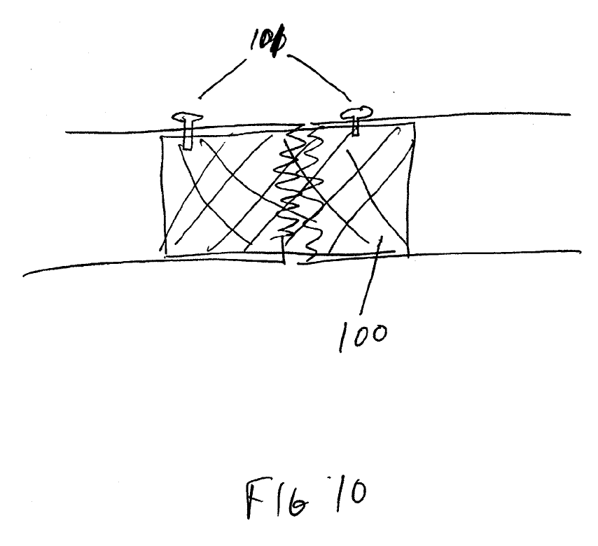

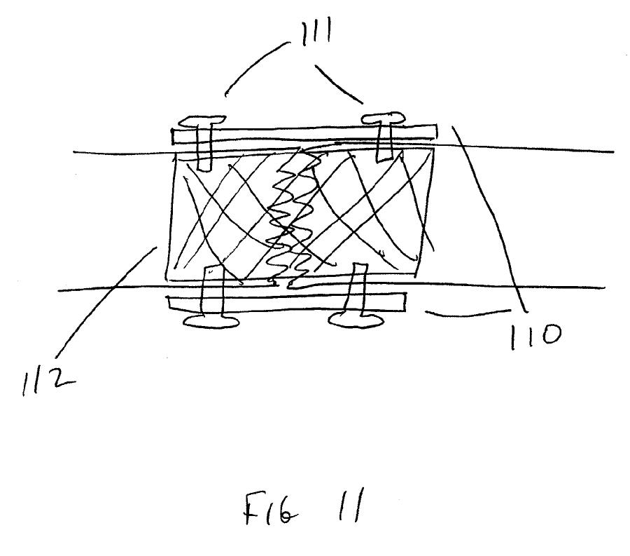

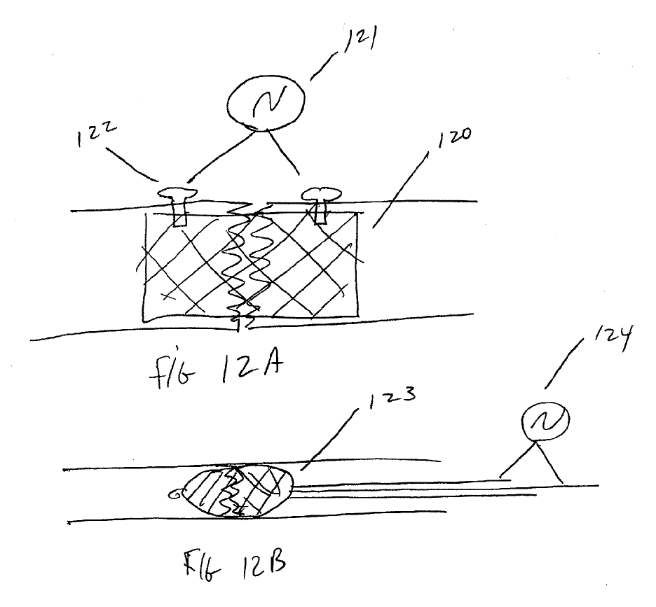

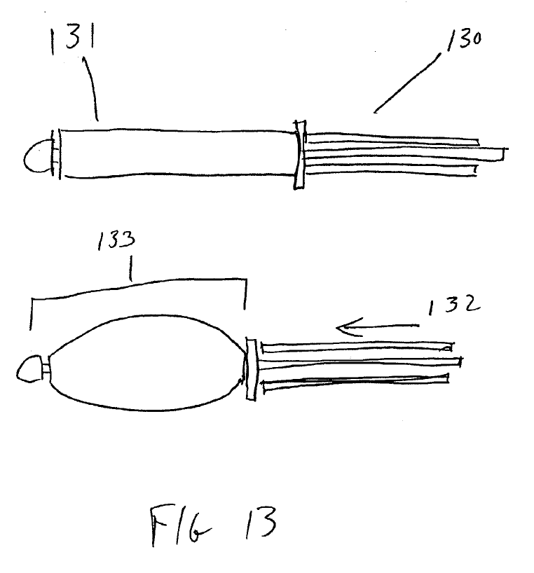

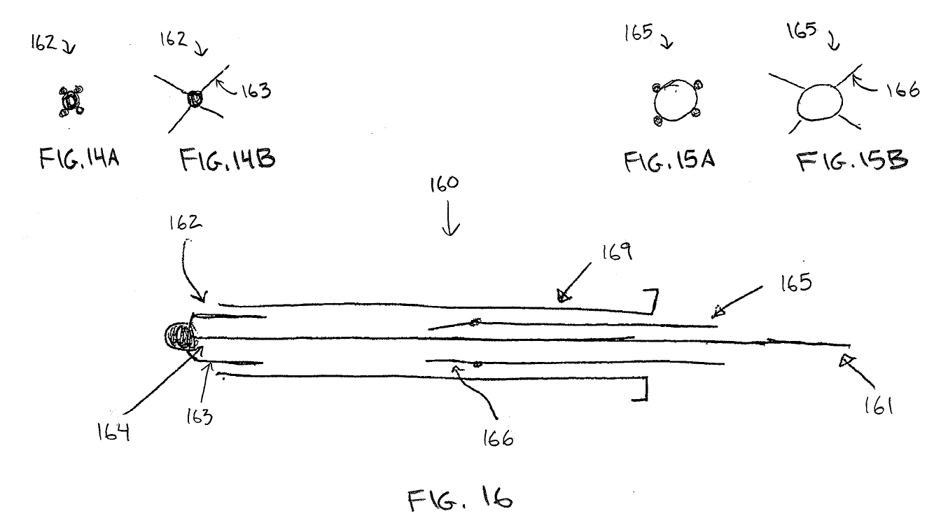

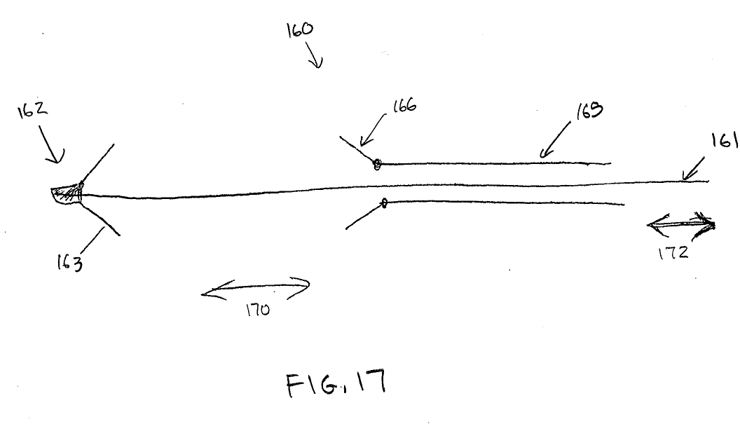

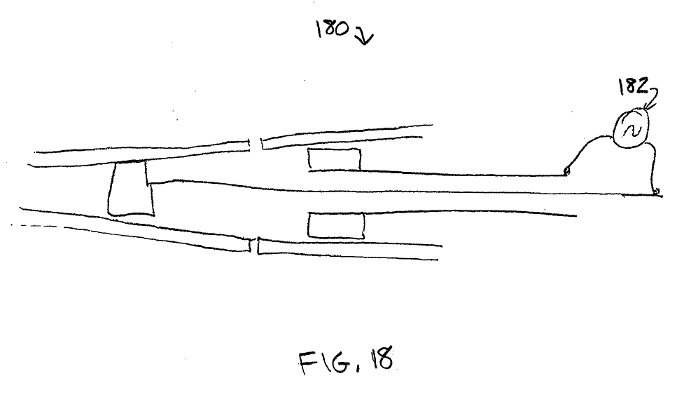

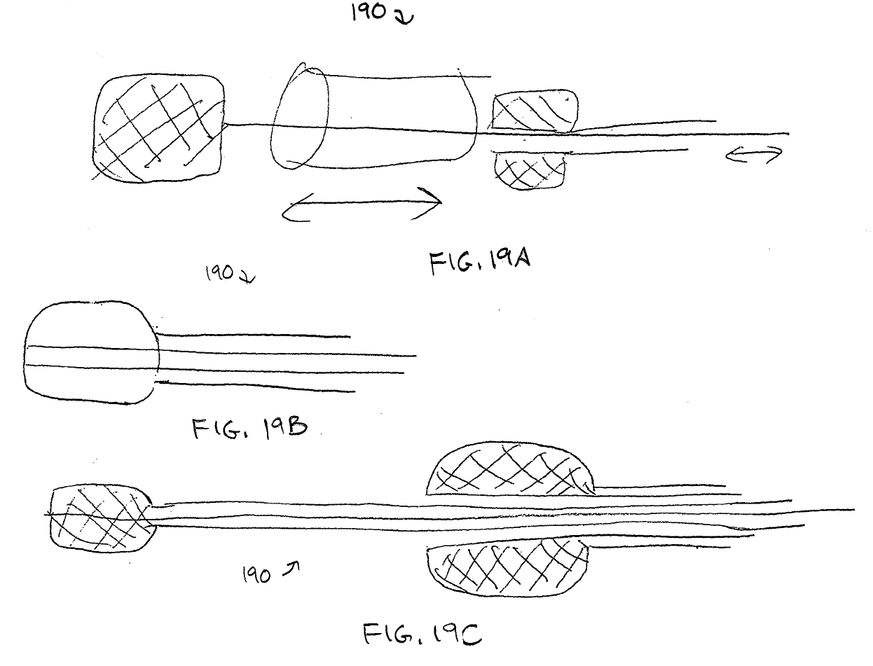

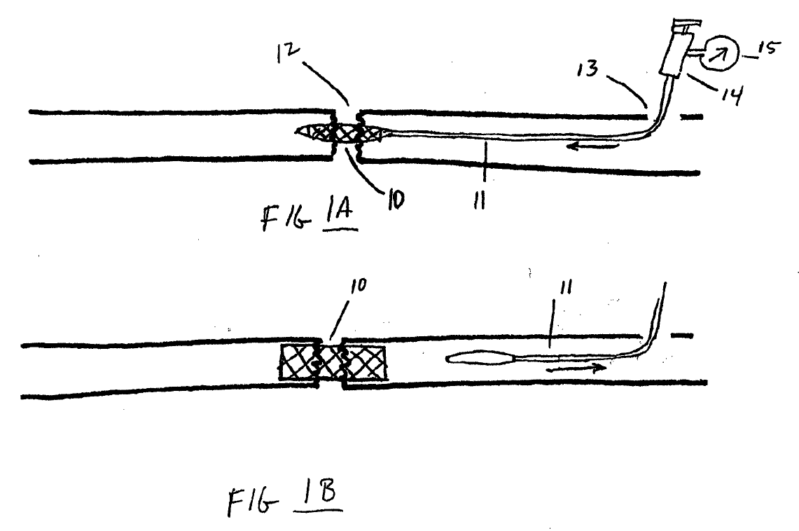

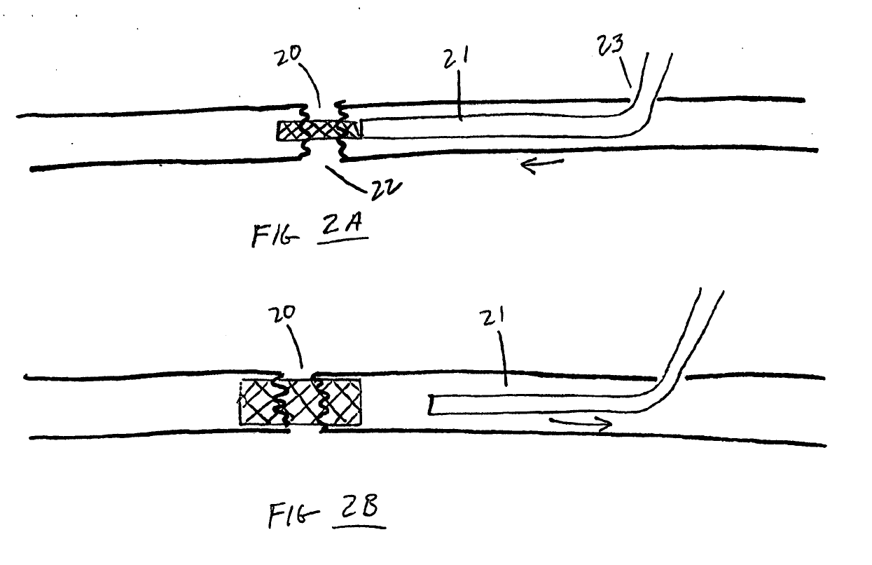

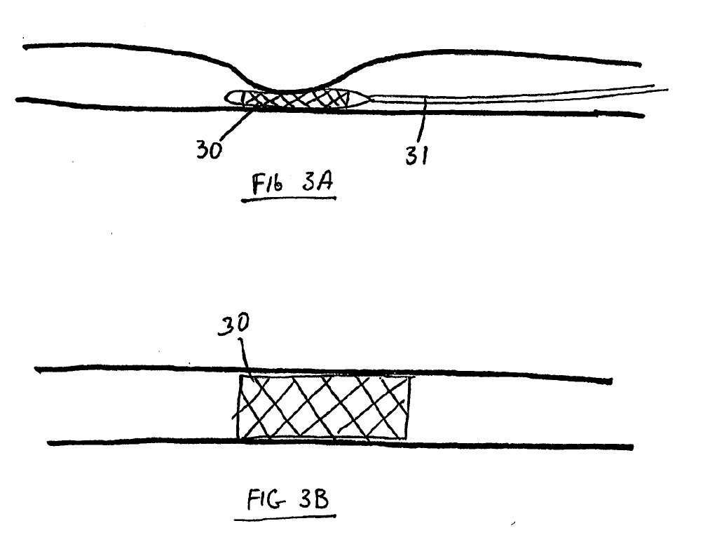

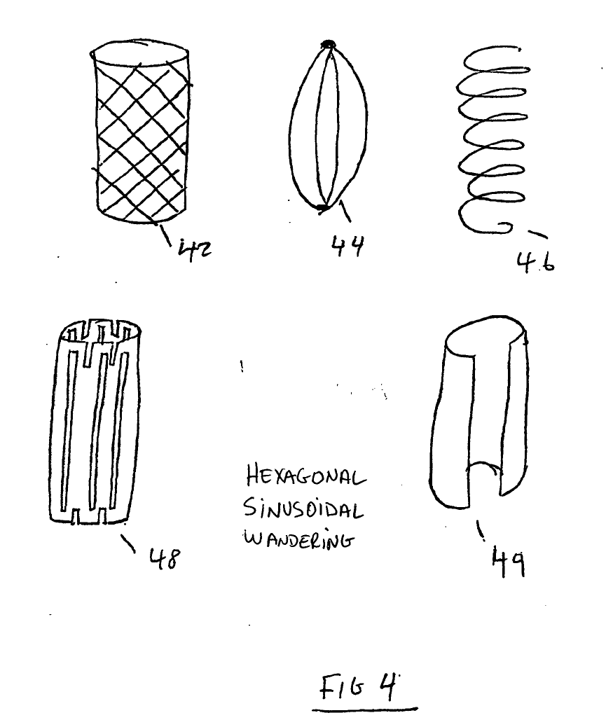

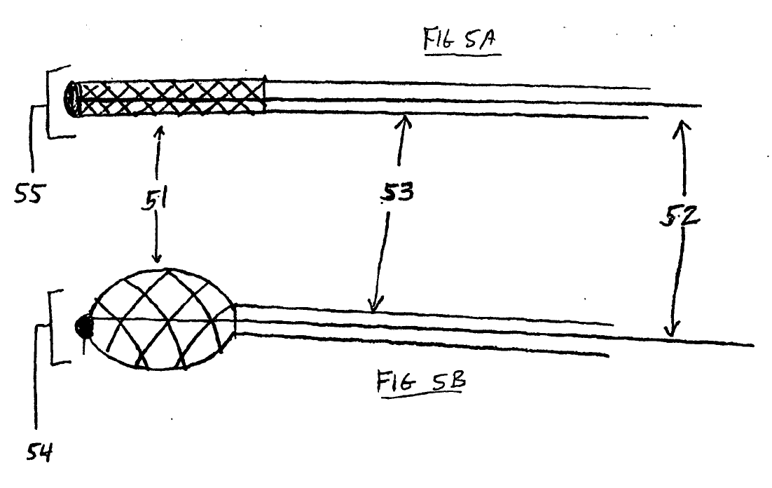

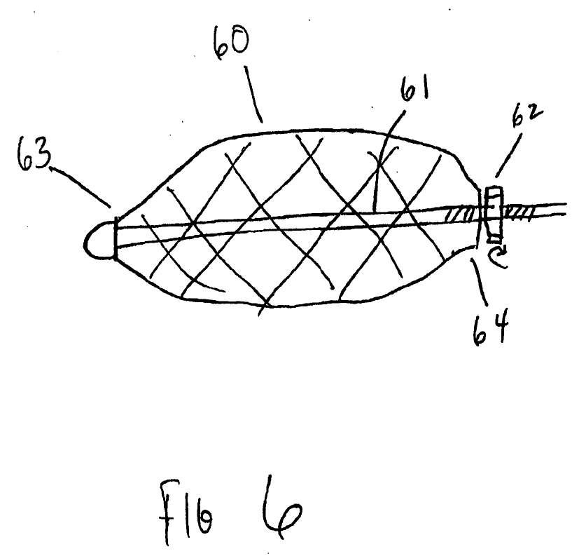

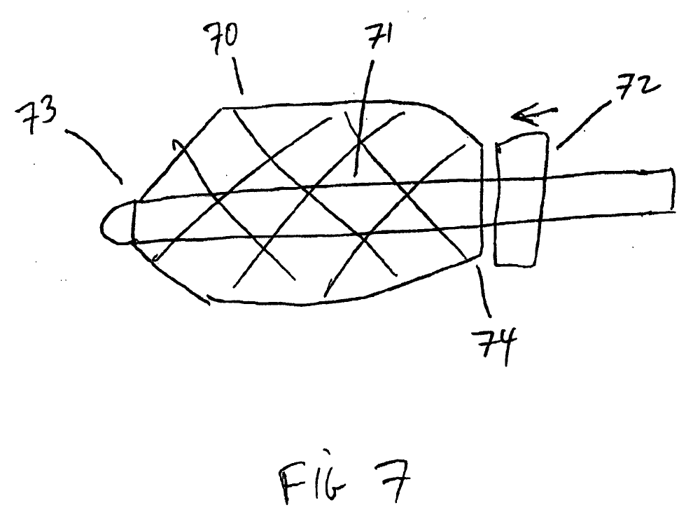

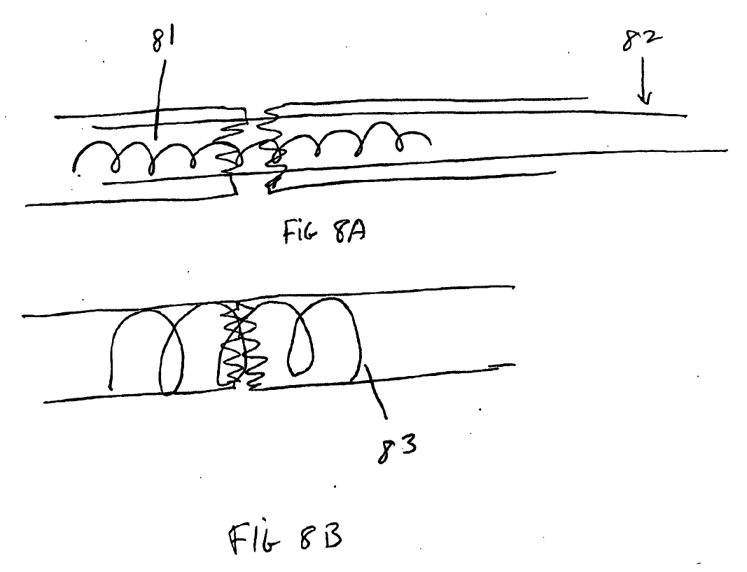

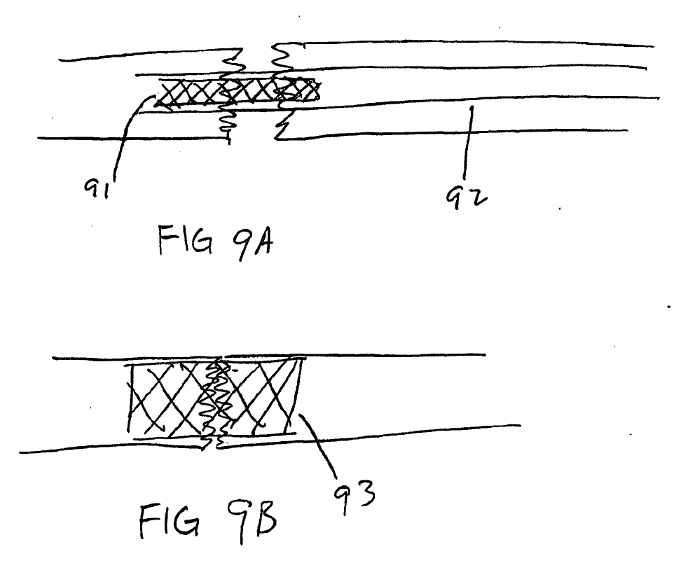

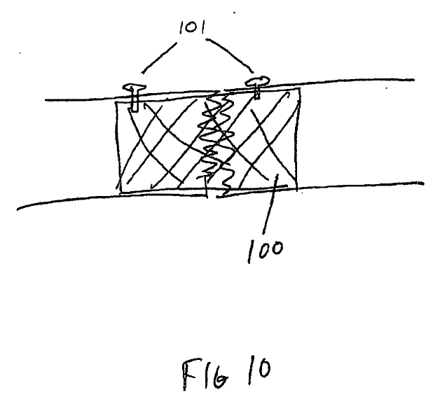

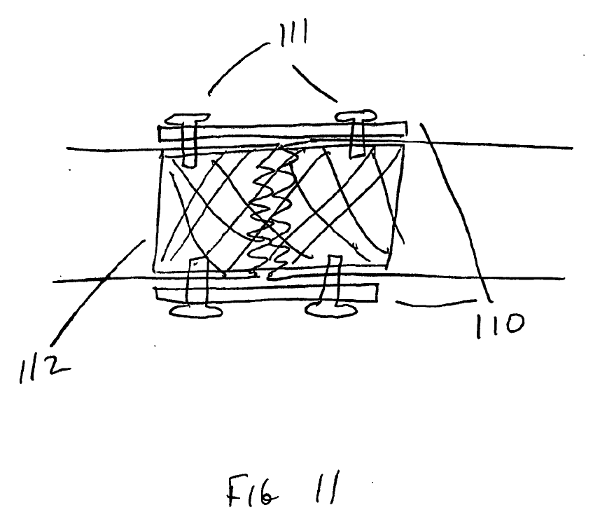

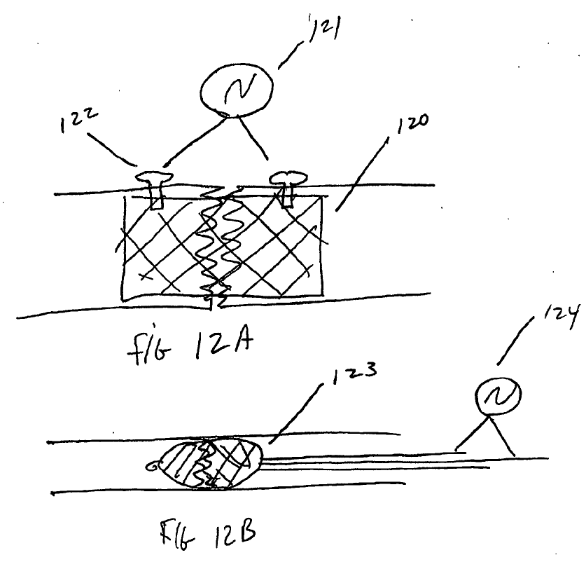

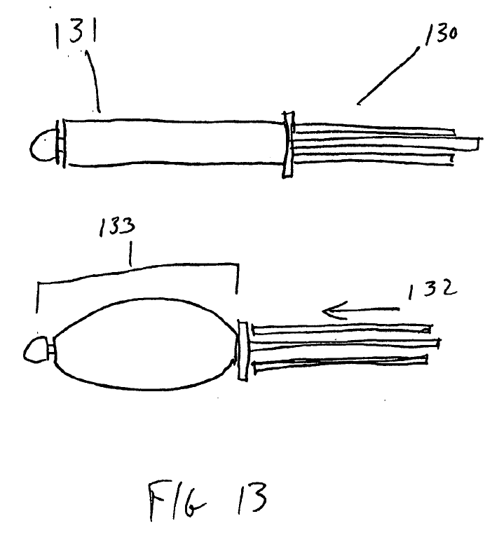

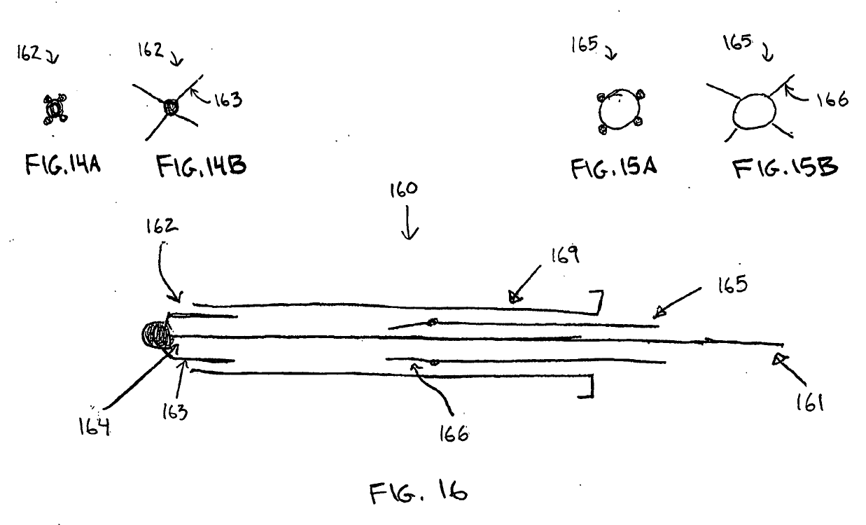

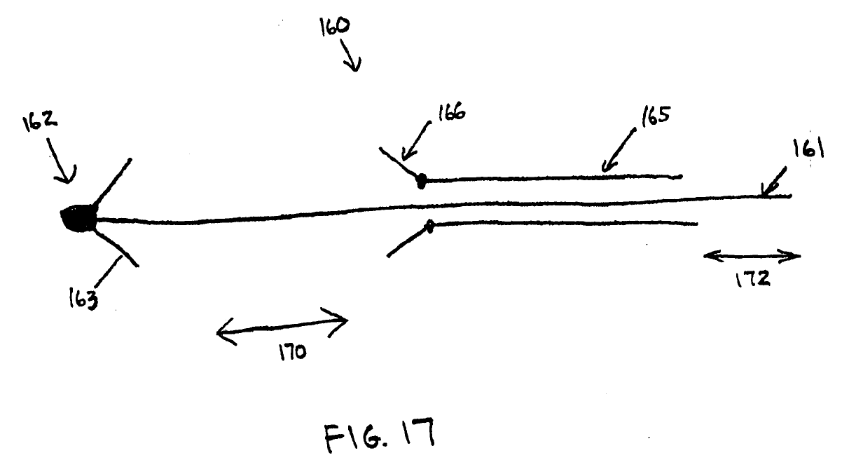

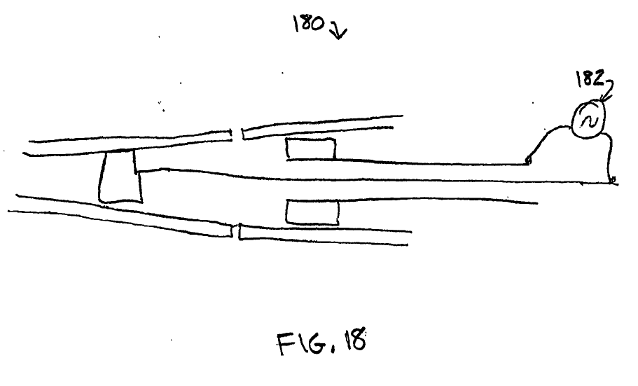

In some embodiments, devices 180 similar to the bone bridge 160 can be attached to an electrical generator 182 to provide electrical stimulation therapy, as is illustrated in FIG. 18. In this scenario, the structures would be electrically insulated form each other. The structures could be connected to an electrical generator 182 in a fashion where one element becomes a cathode and the other an anode. This would provide for a bipolar therapy across the fracture site. Similarly, each electrode could be connected to the same polarity terminal on the generator, while a grounding pad is connected to the other terminal. This would provide therapy in a fashion that is generally known as monopolar. The anchors can be a portion of electrical electrodes that provide electrical stimulation to enhance bone growth or repair.

In some embodiments, the bone bridge may also be constructed of materials that are heated when affected by electrical, laser, RF, magnetic, chemical or other means. This can provide a means of treating cancer or other ailments within the bone.

In some embodiments, the bone bridge can also be cooled cryogenically, by chemical reaction, thermoelectrically. This can provide a means of treating cancer or other ailments within the bone.

In some embodiments, bone bridge designs can be designed to elongate as the natural bone/bone plates growth. This is significantly important for younger people who are still in the growth stages. An example is a coiled bone bridge that is rigid and strong enough to repair the fracture yet also can stretch with the bone growth. Another example is an embodiment of the two structure bone bridge 160. The interlocking locating can designed to allow relative movement of the two structures as the bone grows. Additionally, other methods and designs can be used to cause the bone bridge to elongate. Such methods and designs include heating or cooling of the bone bridge. Materials such as shape-memory metals or plastics can be incorporated into the bone bridge. When heated or cooled, they can contract or elongated. Also, when Nitinol is cooled, it becomes pliable and deformable. Additionally, the bone bridge designs can have mechanical components that are activated with by heating, cooling, RF, magnetically, and other means. These components can form ratcheting elements, threaded rods, bushings, detents, and other elements that when activated, result in an elongation of the BE. Likewise, any of the aforementioned methods creating elongation can also be configured to cause shortening if so needed.

In some embodiments, biological mediators can be applied on or into the bone bridge to improve or enhance the healing process of the fractures. These mediators can include genetically altered osteoblasts, bone morphogenetic proteins, bone graft or bone graft substitutes, artificial or biological osteoconductive matrices, or osteoinductive chemicals. Other materials such as antibiotics or other pharmaceuticals can also be delivered directly to the fracture site via the bone bridge.

In some embodiments, bone bridge methods and constructions can allow for encapsulation of the bone bridge by the new bone growth. Likewise, if so desired, encapsulation may be prevented by materials selection and/or additives to the bone bridge structures.

In some embodiments, as previously disclosed in the above referenced applications, cements, glues, and other substances can be inserted with the bone bridge within the intramedullary space. The substances can be inserted within the entire available intramedullary space or only within the segment occupied by the bone bridge, as well as in one or more discrete segments within the space. This allows for custom therapy and substance placement. These substances can be contained by the bone bridge or portions of the bone bridge as predetermined by the design. As seen in the above referenced applications, a number of the design embodiments of the bone bridge can be used to contain the cements, glues, and other substances within the bone bridge segment. Design modifications can also allow for containment in the distal and/proximal segments of the bone bridge.

In some embodiments, the bone bridge can be made of a material(s), or coated with substances, which result in an intimate bonding or joining of the bone bridge with cements, glues, or other substances that are insert or injected into the intramedullary space.

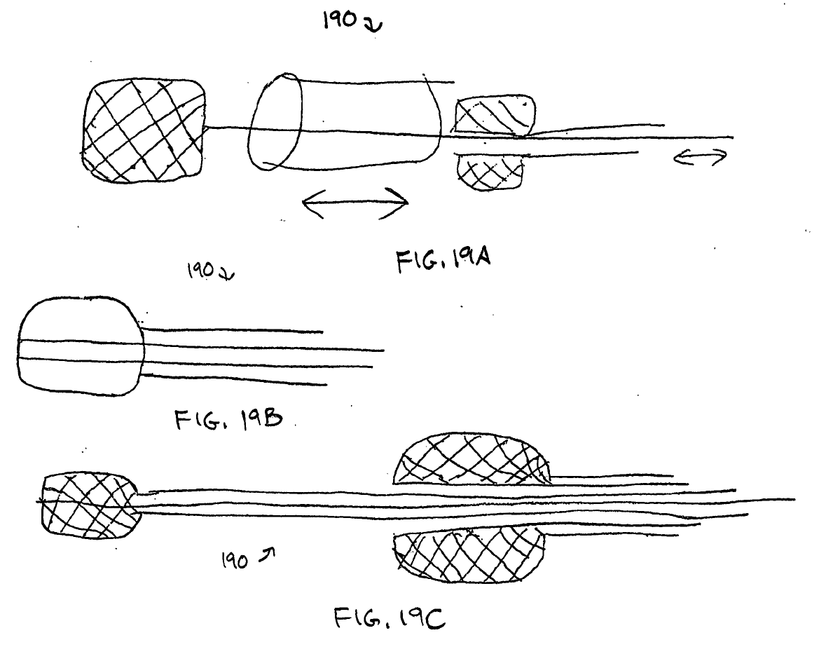

In some embodiments, as previously disclosed in the above referenced applications, cements, glues, and other substances can be inserted with the bone bridge within the intramedullary space. An alternate device design and method includes inserting these substances in a fashion where the resultant is a tubular form of the substances. The tubular form maintains an intramedullary space for bone marrow deposition or formation. The tubular form can be deposited by using a deliver system similar to that disclosed for vascular applications in the U.S. Pat. No. 5,792,106 by Mische. Another method can utilize a delivery system 190 with two or more expandable features (i.e. balloons, braids, restrictors, etc) that create an isolated longitudinal space, as illustrated in FIGS. 19A-19C. Within this space, the substances are injected and solidified. The delivery system is removed after solidification. In another embodiment, this tubular form can actually be preformed of a material that expands diametrically upon hydration, chemical reaction, heat, cold, RF, magnetics, or other interaction. This particular form would have a small diameter to allow for easy insertion into the intramedullary space. This particular expanding tubular form can also be incorporated onto the inside or the outside of the bone bridge, as well as at with or both ends of the bone bridge. The tubular structures would preferably solidify to a state where they provide some mechanical strength. An alternate embodiment would be to eliminate the use of the bone bridge entirely and allow for the afore-mentioned tubular structures to be the only structure implanted within the intramedullary space.

In some embodiments, external fixation devices that wrap around the external surface of the bone can be used. Such as in the case of a longitudinal slit tube or clam-shell type structure that is positioned around the bone at the fracture site. Something similar to this has been previous disclosed in an above referenced application. The devices can be secured or tightened at the slit location by suturing or corseting, clasps, screws and other securement methods. These devices can all be used separately or in conjunction with the other devices and technologies discussed above. Addition of adhesives, cements, or other substance to the inside and outside surfaces of this type of device can provide additional therapeutic benefit. The interior surfaces can have features such as spikes, rough surfaces, grooves, threaded surfaces, etc to help secure and prevent movement.

It should be apparent that various modifications might be made to the devices and methods by one of ordinary skill in the art, without departing from the scope or spirit of the invention.

What is claimed is:

1. A bone bridge for treating bone fractures comprising:
a distal anchor shaft connected to a distal anchor with one or more distal anchor elements configured to expand radially outwardly when deployed; and
a proximal anchor shaft connected to a proximal anchor with one or more proximal anchor elements configured to expand radially outwardly when deployed, where the distal anchor shaft and the proximal anchor shaft are longitudinally moveable relative to each other.
